(19) United States
(12) Patent Application Publication
(10) Pub. No.: US 2021/0393842 A1
(43) Pub. Date: Dec. 23, 2021

(54) Face mash

(71) Applicant: Ernesto Padilla, El Paso, TX (US)

(72) Inventor: Ernesto Padilla, El Paso, TX (US)

(21) Appl. No.: 17/300,399

(22) Filed: Jun. 14, 2021

Related U.S. Application Data
(60) Provisional application No. 63/102,540, filed on Jun. 17, 2020.

(51) Int. Cl. A61L 9/20; A62B 18/02; A62B 18/08; A62B 7/10

(52) U.S. Cl. CPC A61L 9/20; A62B 18/025; A61L 2209/14; A62B 7/10; A62B 18/08

(57) ABSTRACT

A facial mask for protection from airborne contagions utilizing ultraviolet light as well as mechanical filtration.

### BACKGROUND OF THE INVENTION

### 1. Field of the Invention

This invention relates to an improved face mask.

### 2. Description of the Prior Art

A face mask worn to cover a person's mouth and nose in order to prevent the inhalation or expulsion of contagions has been in use for at least 400 years. While, in general, face masks consists of a cloth material of sufficient size to cover the nose and mouth of an individual, removably attached to such individual's right and left ear, the shape and material construction of such mask has definitely evolved. In 17^{th} century Europe, plague doctors used a mask that resembled a long, pointed beak filled with perfumes and aromatics that masked bad odors. Cotton filters worn over the mouth and nose first appeared around the 19 century, however, their use was not widespread. During the 1910 epidemic of pneumonia plague in Manchuria, China, cotton masks were used by medical personnel and the general public to reduce the spread of the disease. Medical personnel as well as the general public in the United States first began widespread use of the cotton mask during the 1918 flue epidemic. In the early 21^{st} century, masks were designed as fashionable accessories to primarily block out smog and dust particles. It has become increasingly apparent during the current COVID 19 pandemic, that a face mask constructed of only cloth material is not 100% effective in eliminating contagions from either entering or leaving the masks and can be uncomfortable to be worn.

Ultraviolet (“UV”) light is electromagnetic radiation with wavelengths shorter than visible light but longer than X-Rays. Short-wavelength UV light between 200 nm and 300 nm is considered germicidal UV. Ultraviolet germicidal irradiation (“UVGI”) has been an accepted practice since the mid-20th century. At these wavelengths, the energy is stronbly absorbed by nucleic acids. The absorbed energy results in defects within the organization that prevent replication or the expression of necessary proteins resulting in the death or inactivation of the microorganism. Utilizing UVGI or any other light source which will destroy or eliminate contagions with a facial mask will increase the effectiveness of such mask.

### SUMMARY OF THE INVENTION

It is, therefore, an object of the present invention to provide a facial mask that incorporates the use of a light source such as UVGI to destroy or eliminate contagions. Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is an exploded view of the mask of the present invention.

### DESCRIPTION OF THE PREFERRED EMBODIMENTS

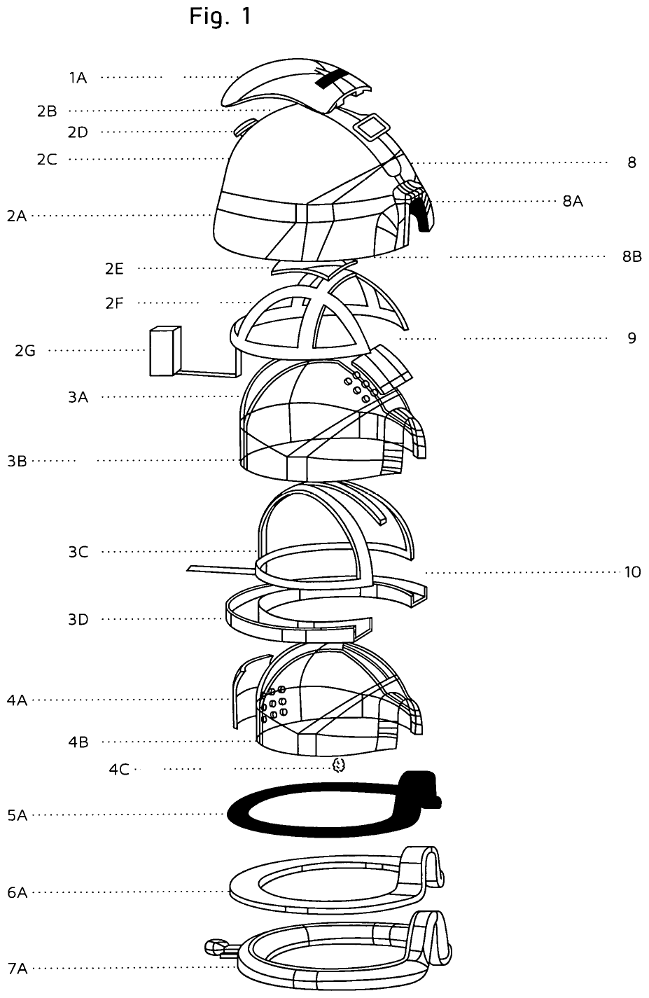

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention. As can be seen in FIG. 1, the mask [8] of the present invention includes an outside surface [8A] and inner surface [8B] which comfortably fits against a person's face through use of an inflatable gasket [7A]. To help eliminate particles, the mask [8] can be made of material with electrostatic properties. The inflatable gasket [7A] includes a manual pump with relief valve so that the air pressure within such gasket can be customized to each individual user. The mask [8] can be made of any suitable material of sufficient rigidity and strength to support the various components contained therein such as plastic or silicone. The UVGI [30] can be any light source which emits light in a wavelength that will damage or eliminate any contagion entering the mask [8]. Shown in FIG. 1 is a light emitting diode strip [2F] that attaches to the inner surface [8B] of the mask[8]. Any power source sufficient to power the UVGI such as a rechargeable battery pack [2G] can be used. The battery pack [2G] can be recharged using any conventional means such as solar power or standard electrical wall outlet. The inner surface [8B] of the mask [8] can include a reflective surface to improve UVGI efficiency. Various mechanical blockages to air flow in or out of the mask can be present within the mask [8] such as a front shield [1A], or at least one filtering membrane. The mask [8] of the present invention as shown in FIG. 1 depicts the use of 4 filtering membranes [2E], [3A], and [4A].

While not necessary to embodiment, the mask [8] shown in depicts two separate chambers, chamber 1 [9] and chamber 2 [10], each with a light source [2F] and [3C] respectively. Although not necessary for efficient elimination of contagions, to improve the ability to communicate while wearing the mask [8], a microphone [4C] with speaker [2B] can be added. Additionally, shown in FIG. 1 is a hazardous gas/microbe detector with alert system [20] and an access point for use of a beverage straw [2D].

While the preferred embodiments have been shown and described, it will be understood that there is no intent to limit the invention by such disclosure, but rather, is intended to cover all modifications and alternate constructions falling within the spirit and scope of the invention as defined in the appended claims.

1. A facial mask containing at least one chamber with at least one light source which will destroy or eliminate contagions.

2. The facial mask of claim 1 wherein the light source is short wavelength ultraviolet light.

3. The facial mask of claim 2 wherein the wavelength of ultraviolet light is between 200 nm and 300 nm.

4. A facial mask containing at least one chamber with at least one light source which will destroy or eliminate contagions further comprising an inner and outside surface.

5. The facial mask of claim 4 wherein the inner surface further comprises an inflatable gasket along the edges of said inner surface.

6. The facial mask of claim 4 wherein the light source is ultraviolet light.

7. The facial mask of claim 6 wherein the wavelength of the ultraviolet light is between 200 nm and 300 nm.

8. The facial mask of claim 6 wherein the source of the ultraviolet light is a light emitting diode.

9. The facial mask of claim 8 wherein the light emitting diode is rechargeable.

10. A facial mask containing at least one chamber with at least one light source which will destroy or eliminate contagions further comprising an inner and outside surface.

11. The facial mask of claim 10 wherein the inner surface further comprises an inflatable gasket along the edges of said inner surface.

12. The facial mask of claim 10 wherein said inner surface is coated with a reflective surface.

13. The facial mask of claim 10 wherein at least one filtering membrane is included.

14. The facial mask of claim 10 wherein said mask is made from material with electrostatic properties.

15. The facial mask of claim 10 further comprising a microphone and speaker to facilitate communication through said mask.

16. The facial mask of claim 10 further comprising a hazardous gas/microbe detector with audible and visual alert system.

17. The facial mask of claim 10 further comprising an access point through said mask for a straw.
